(19) United States
(12) Patent Application Publication
(10) Pub. No.: US 2023/0005577 A1
(43) Pub. Date: Jan. 5, 2023

(54) HEALTH DATA MANAGEMENT SYSTEM, HEALTH DATA MEASUREMENT APPARATUS, AND HEALTH DATA MANAGEMENT METHOD

(71) Applicants: Hiroyuki Sugiyama, Kanagawa (JP); Taira Otsuka, Kanagawa (JP); Hiroki Atari, Kanagawa (JP); Yasunobu Shimizu, Kanagawa (JP); Chikara Hiraoka, Kanagawa (JP)

(72) Inventors: Hiroyuki Sugiyama, Kanagawa (JP); Taira Otsuka, Kanagawa (JP); Hiroki Atari, Kanagawa (JP); Yasunobu Shimizu, Kanagawa (JP); Chikara Hiraoka, Kanagawa (JP)

(73) Assignee: Ricoh Company, Ltd., Tokyo (JP)

(21) Appl. No.: 17/804,649

(22) Filed: May 31, 2022

(30) Foreign Application Priority Data
Jul. 5, 2021 (JP) 2021-111390

(51) Int. Cl. G16H 10/60; A61B 5/00; G16H 50/20; G16H 50/30; G16H 50/70

(52) U.S. Cl. CPC G16H 10/60; A61B 5/743; G16H 50/20; G16H 50/30; A61B 5/0004; G16H 50/70

(57) ABSTRACT

A health data management system includes a health data measurement apparatus and a health management server communicable therewith. The apparatus includes medical and health equipment that measures health data of a user and first circuitry that collects the health data. The management server includes a first memory that stores the health data from the apparatus and second circuitry. Based on the stored health data, the second circuitry generates health management data representing a health state of the user, and analyzes at least one of an item indicating a worsening health state of the user, an item with insufficient measurement data, or an item lacking measurement data as a required check item. When determined that the health management data includes the required check item, the second circuitry transmits to the apparatus a message prompting the user to take a measurement of the required check item, and requests the health data.

### CROSS-REFERENCE TO RELATED APPLICATION

This patent application is based on and claims priority pursuant to 35 U.S.C. § 119(a) to Japanese Patent Application No. 2021-111390, filed on Jul. 5, 2021, in the Japan Patent Office, the entire disclosure of which is hereby incorporated by reference herein.

### BACKGROUND

### Technical Field

The present invention relates to a health data management system, a health data measurement apparatus, and a health data management method.

### Description of the Related Art

A typical company or organization provides employees with periodic medical examination at least once a year. It would be more desirable if the company or organization introduces a more accessible health management system suited to the lifestyles of the individual employees to enable the employees to manage their health in daily life.

The office of the company or organization is typically installed with a multifunction peripheral/product/printer (MFP); the MFP has become a familiar apparatus used by many users. A health management system utilizing the MFP therefore would enable health management in daily life.

Further, collecting health management data via MFPs and storing the collected health management data in bulk enables statistical data aggregation, which is expected to contribute to more advanced health management.

A health management system using functions of the MFP installed in an office, for example, is capable of storing and outputting health data voluntarily measured by users.

### SUMMARY

In one embodiment of this invention, there is provided a health data management system that includes, for example, a health data measurement apparatus and a health management server. The health data measurement apparatus includes medical and health equipment and first circuitry. The medical and health equipment measures health data related to a body of a user using the medical and health equipment. The first circuitry collects the health data of the user from the medical and health equipment. The health management server is communicable with the health data measurement apparatus, and includes a first memory and second circuitry. The first memory stores the health data of the user from the health data measurement apparatus. Based on the health data of the user stored in the first memory, the second circuitry generates health management data representing a health state of the user. Based on the generated health management data, the second circuitry analyzes at least one of an item indicating a worsening health state of the user, an item with insufficient measurement data, or an item lacking measurement data as a required check item. Based on the analysis, the second circuitry determines whether the health management data includes the required check item. Based on a determination that the health management data includes the required check item, the second circuitry transmits to the health data measurement apparatus a message prompting the user to take a measurement of the required check item, and requests the health data measurement apparatus to transmit the health data of the user collected from the medical and health equipment to the health management server.

In one embodiment of this invention, there is provided a health data measurement apparatus connected to a health management server via a network. The health data measurement apparatus includes, for example, a display, medical and health equipment, and circuitry. The medical and health equipment measures health data related to a body of a user. The circuitry controls measurement of the medical and health equipment. In response to receipt of a request from the health management server, the circuitry controls the display to display a message prompting the user to take a measurement of a required check item, and transmits the health data of the user measured by the medical and health equipment to the health management server. The required check item is at least one of an item indicating a worsening health state of the user, an item with insufficient measurement data, or an item lacking measurement data.

In one embodiment of this invention, there is provided a health data management method that includes, for example, storing health data of a user in a memory from a health data measurement apparatus. The health data of the user is collected to the health data measurement apparatus from medical and health equipment. The health data management method further includes generating, based on the health data of the user stored in the memory, health management data representing a health state of the user, analyzing, based on the generated health management data, at least one of an item indicating a worsening health state of the user, an item with insufficient measurement data, or an item lacking measurement data as a required check item, determining, based on the analysis, whether the health management data includes the required check item, and based on a determination that the health management data includes the required check item, transmitting to the health data measurement apparatus a message prompting the user to take a measurement of the required check item, and requesting the health data measurement apparatus to transmit the health data of the user collected from the medical and health equipment to the health management server.

### BRIEF DESCRIPTION I/F THE SEVERAL VIEWS I/F THE DRAWINGS

A more complete appreciation of the disclosure and many of the attendant advantages and features thereof can be readily obtained and understood from the following detailed description with reference to the accompanying drawings, wherein:

FIG. 1 is a diagram illustrating a configuration of a health management system according to a first embodiment of the present invention;

FIG. 2 is a block diagram illustrating a hardware configuration of a health data measurement apparatus included in the health management system of the first embodiment;

FIG. 3 is a diagram illustrating a hardware configuration of a health management server included in the health management system of the first embodiment illustrated in FIG. 1;

FIG. 4 is a block diagram illustrating a functional configuration of the health management system of the first embodiment;

FIG. 5 is a flowchart illustrating an operation of the health management system of the first embodiment;

FIG. 6 is a diagram illustrating a series of user interfaces displayed on a front surface of the health data measurement apparatus in the first embodiment to prompt a user to take a measurement of body composition based on a determination that the measurement by the user is necessary, and guide the user to complete the measurement;

FIG. 7 is a diagram illustrating a series of user interfaces displayed on the front surface of the health data measurement apparatus in a second embodiment of the present invention to notify the user of a determination that the measurement of an item measurable by the user with a smartwatch is necessary and guide the user to complete data reading; and

FIG. 8 is a diagram illustrating a series of user interfaces displayed on the front surface of the health data measurement apparatus in a third embodiment of the present invention to guide the user to use the health data measurement apparatus when there is no item for which the measurement or data reading is necessary.

The accompanying drawings are intended to depict embodiments of the present invention and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to be limiting of the present invention. As used herein, the singular forms “a”, “an” and “the” are intended to include the plural forms as well, unless the context clearly indicates otherwise. In the drawings illustrating embodiments of the present invention, members or components having the same function or shape will be denoted with the same reference numerals to avoid redundant description.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

In an embodiment of the present invention, a health data management system (hereinafter simply referred to as the health management system) displays a message prompting a user to take a measurement of a required check item of health management data related to the body of the user.

Embodiments of the present invention will be described in detail with reference to the accompanying drawings. Components described in the embodiments and the types, combinations, shapes and relative positions of the components are illustrative and should not be interpreted to limit the scope of the present invention, unless otherwise specified.

A configuration of a health management system according to a first embodiment of the present invention will be described.

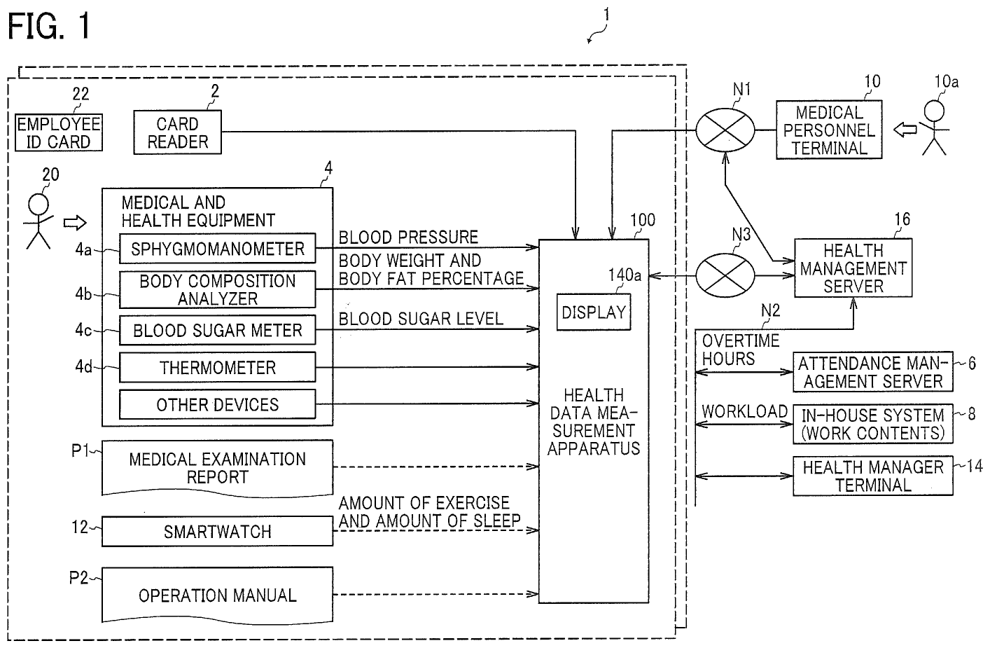

FIG. 1 is a diagram illustrating a configuration of the health management system of the first embodiment. As illustrated in FIG. 1, a health management system 1 includes a card reader 2, medical and health equipment 4, an attendance management server 6, an in-house system 8, a medical personnel terminal 10, a smartwatch 12, a health manager terminal 14, a health management server 16, and a health data measurement apparatus 100.

The card reader 2 reads a unique employee identifier (ID) from an employee ID card 22 possessed by a user 20, and outputs the read employee ID to the health data measurement apparatus 100. In response to receipt of the employee ID from the card reader 2, the health data measurement apparatus 100 transmits the employee ID and an authentication request to the attendance management server 6, and receives an authentication result from the attendance management server 6.

The medical and health equipment 4 is connected to devices such as a sphygmomanometer 4a, a body composition analyzer 4b, a blood sugar meter 4c, and a thermometer 4d. Health data obtained from these devices of the medical and health equipment 4 is processed by the health data measurement apparatus 100.

The attendance management server 6 manages working hours data of the individual user 20. The attendance management server 6 further stores work management data, which is updated daily.

The in-house system 8 inside a company manages work contents of the individual user 20 (i.e., employee).

The medical personnel terminal 10 (i.e., a first terminal) is a personal computer (PC) connected to the health data measurement apparatus 100 via an external network N1 outside the company. The medical personnel terminal 10 is operated by medical personnel 10a who provide explanations of the medical and health equipment 4.

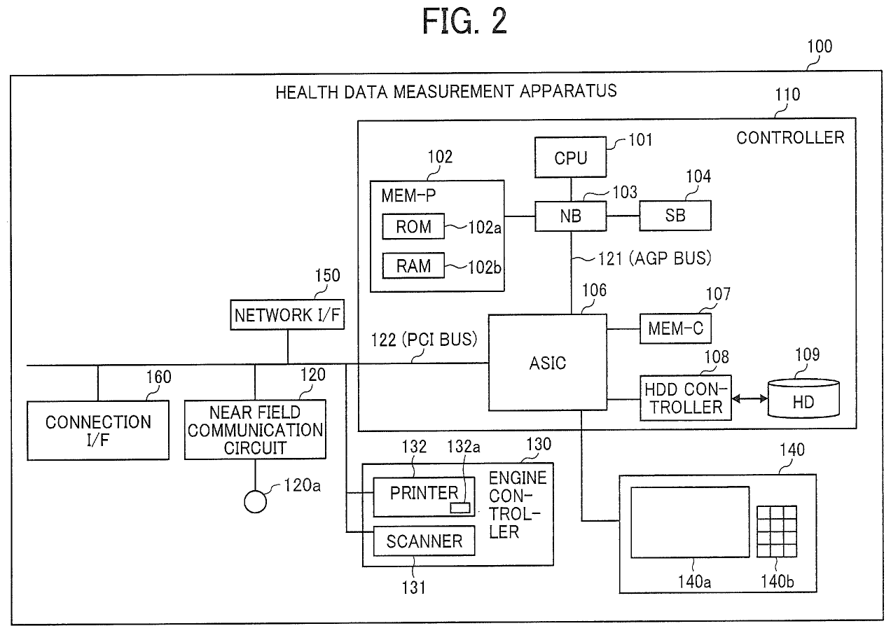

The health data measurement apparatus 100 reads a past medical examination result of the user 20 with a scanner 131 in FIG. 2, and stores the read medical examination result in a hard disk (HD) 109 in FIG. 2 and the health management server 16.

The smartwatch 12 includes an optical sensor, a contact pressure sensor, an acceleration sensor, and a global positioning system (GPS) sensor. The smartwatch 12 calculates the health data based on data acquired from a human wrist with these sensors.

The optical sensor irradiates a wrist of the user 20 with light emitting diode (LED) light, receives the light reflected from the wrist, and calculates the blood pressure and pulse of the user 20 based on the level of the received reflected light.

The contact pressure sensor detects the pressure of an area of a human body in contact with the contact pressure sensor, and a central processing unit (CPU) calculates the blood pressure and pulse based on the value of the contact pressure sensor.

The acceleration sensor detects the acceleration rates in three axial directions, for example.

The GPS sensor calculates positional information of a reception point based on radio signals received from multiple GPS satellites.

The smartwatch 12 calculates the blood pressure and pulse with sensors such as the optical sensor and the contact pressure sensor. The smartwatch 12 further calculates the amount of activity based on the value of the acceleration sensor and the positional information of the user 20, and measures the length and depth of sleep based on statistical results obtained with the above-described sensors. The smartwatch 12 stores the health data (e.g., the blood pressure, the changes in the pulse, the amount of activity, and the length and depth of sleep) of the user 20, and displays the stored health data of the user 20 as a two-dimensional code.

Specifically, the smartwatch 12 calculates the blood pressure by combining the measurement result of the heart rate measured based on electrical signals and the measurement result of the blood flow rate measured with the optical sensor. The smartwatch 12 includes a light emitting device and a light receiving device. With the light emitting device irradiating the wrist of the user 20 with light and the light receiving section detecting the light reflected from a blood vessel in the wrist, the smartwatch 12 performs optical calculation to measure the blood pressure. The smartwatch 12 further detects the sleep based on the pulse and body movements and calculates the length and depth of the sleep. The smartwatch 12 is capable of detecting movement and thus calculating the amount of activity.

Any desired configuration of the smartwatch is applicable to the smartwatch 12. For example, the configurations of the smartwatch disclosed in Japanese Unexamined Patent Application Publication Nos. 2018-191785, 2018-191787, 2019-141405, and 2014-223098 are applicable to the smartwatch 12.

The health manager terminal 14 (i.e., a second terminal) is a PC connected to an in-house network N2. The health manager terminal 14 is operated by a health manager who is engaged in the health management of the user 20 (i.e., employee).

The health management server 16 is a computer server connected to the in-house network N2. The health management server 16 acquires the health data of the user 20 (i.e., employee) and manages the acquired health data of the user 20 in association with the corresponding employee ID.

The health data measurement apparatus 100 is a multifunction peripheral/product/printer (MFP). A single MFP functions as a printer, a scanner, a copier, and a facsimile (FAX) machine, for example.

The health data measurement apparatus 100 cooperates with the attendance management server 6 inside the company, and thus is able to obtain the work contents of the individual user 20 from work records. The health data measurement apparatus 100 is therefore capable of providing appropriate health support to the user 20 by recognizing the correlation between the health condition and the work conditions based on the workload and working hours, in addition to the data measured with the medical and health equipment 4 attached to the health data measurement apparatus 100, the information of past medical examinations, and the data of the daily state of health obtained from the smartwatch 12. For example, if the correlation between a reduction in sleeping hours and an increase in working hours is recognized, the health data measurement apparatus 100 is capable of sending the user or the supervisor of the user 20 a recommendation to reduce the working hours.

MFPs or printers are typically connected to each other via a network, and information of the MFPs or printers is transmitted to a server of the vender of the MFPs or printers. Specifically, information such as the usage rates of toner and supply units, the notifications of the replacement times of supplies, the usage patterns of users (e.g., patterns related to the print volume, the image area, the color or monochrome setting information, and the use of staples), and environment information is transmitted to a server installed in a call center, for example. Such information is stored as big data and used widely in the operations of a printer design section, a failure prediction section, and other sections of the company.

In the first embodiment, the server connected to the MFPs or printers via a network (i.e., the health management server 6) is used to store the measurement information.

The above-described information is stored in the health data measurement apparatus 100 and the health management server 16 inside the company, enabling the observation over time of the health state of the user 20. Further, if a seemingly anomalous item is detected through the continuous observation, the health management server 16 orders the user 20 to take a measurement of the item when the user 20 uses the health data measurement apparatus 100.

The health management server 16 stores, as well as the data of the user 20 having used the health data measurement apparatus 100, the data received from a plurality of health data measurement apparatuses 100 connected to the network, enabling analysis based on big data. Consequently, the medical personnel 10a are able to examine the big data stored in the health management server 16 to identify a sudden anomaly in an individual, characteristics of the respective sections of the company, or a local epidemic, for example.

Based on the above-described information, the medical personnel 10a are also able to additionally register an item for which the measurement with the health data measurement apparatus 100 should be promoted, and to send a direct instruction to the user 20 or a health manager of the section in which the health data measurement apparatus 100 is installed.

For example, maintaining statistics on body temperature with the health management server 16 enables the identification of the distribution of people with fever, i.e., people with a particular body temperature or higher. Further, comparing the statistics on body temperature with attendance management information contributes to the identification of a region of influenza epidemic, for example.

The health data measurement apparatus 100 is capable of printing the measurement result on a recording medium or outputting the measurement result to a display 140a. Whether to print or display the measurement result may be determined as desired by the user 20.

A configuration of the health data measurement apparatus 100 will be described.

FIG. 2 is a block diagram illustrating a hardware configuration of the health data measurement apparatus 100 of the first embodiment. FIG. 2 illustrates a hardware configuration of an image forming apparatus (an MFP) as an example of the health data measurement apparatus. As illustrated in FIG. 2, the health data measurement apparatus 100 implemented by an MFP includes a controller 110, a near field communication circuit 120, an engine controller 130, an operation panel 140, a network interface (I/F) 150, and a connection I/F 160.

The controller 110 includes a CPU 101 as a major component of a computer forming the health data measurement apparatus 100, a system memory (MEM-P) 102, a north bridge (NB) 103, a south bridge (SB) 104, an application specific integrated circuit (ASIC) 106, a local memory (MEM-C) 107 serving as a storage device, a hard disk drive (HDD) controller 108, and the HD 109 serving as a storage device. The NB 103 and the ASIC 106 are connected to each other by an accelerated graphics port (AGP) bus 121.

The CPU 101 is a control device that performs overall control of the health data measurement apparatus 100. The NB 103 is a bridge for connecting the CPU 101, the MEM-P 102, the SB 104, and the AGP bus 121 to each other. The NB 103 includes a memory controller that controls data writing and reading to and from the MEM-P 102, a peripheral component interconnect (PCI) master, and an AGP target.

The MEM-P 102 includes a ROM 102a and a RAM 102b. The ROM 102a is a memory for storing programs and data for implementing the functions of the controller 110. The RAM 102b is used as a memory in deploying a program or data or as a rendering memory in memory printing, for example. Each of the programs stored in the ROM 102a may be provided as recorded on a computer readable recording medium, such as a compact disc-ROM (CD-ROM), a CD-recordable (CD-R), or a digital versatile disc (DVD), in an installable or executable file format.

The SB 104 is a bridge for connecting the NB 103 to a PCI device or a peripheral device. The ASIC 106 is an integrated circuit (IC) for image processing, which includes hardware components for image processing. The ASIC 106 functions as a bridge for connecting the AGP bus 121, a PCI bus 122, the HDD controller 108, and the MEM-C 107 to each other. The ASIC 106 includes a PCI target, an AGP master, an arbiter as a core component of the ASIC 106, a memory controller that controls the MEM-C 107, a plurality of direct memory access controllers (DMACs) that execute processes such as the rotation of image data with a hardware logic, and a PCI unit that transfers data to and from the scanner 131 or a printer 132 of the engine controller 130 via the PCI bus 122. The ASIC 106 may be connected to a universal serial bus (USB) interface and an institute of electrical and electronics engineers (IEEE) 1394 interface.

The MEM-C 107 is a local memory used as a copy image buffer and a code buffer. The HD 109 is a storage device for storing image data, font data for use in printing, and form data. The HDD controller 108 controls data writing and reading to and from the HD 109 under the control of the CPU 101. The AGP bus 121 is a bus interface for a graphics accelerator card proposed to speed up graphics processing. The AGP bus 121 enables the graphics accelerator card to directly access the MEM-P 102 with a high throughput, thereby implementing a high-speed graphics accelerator card.

The near field communication circuit 120 includes an antenna 120a. The near field communication circuit 120 is a communication circuit conforming to a standard such as near field communication (NFC) or Bluetooth (registered trademark).

The engine controller 130 includes the scanner 131 and the printer 132. The operation panel 140 includes the display 140a and an operation device 140b. The display 140a is implemented by a panel such as a touch panel to display the current setting values and a selection screen and receive an input from an operator (e.g., the user 20). The operation device 140b includes keys such as numeric keys for receiving the setting values of conditions related to image formation such as a density setting condition and a start key for receiving a copy start instruction. The controller 110 controls the entire health data measurement apparatus 100 to control rendering, communication, and input via the operation panel 140, for example. Each of the scanner 131 and the printer 132 includes an image processing section that performs processing such as error diffusion and gamma conversion.

With an application switch key included in the operation panel 140, the function of the health data measurement apparatus 100 is sequentially switched to a document box function, a copier function, a printer function, and a FAX function to select a desired function. When the document box function is selected, the health data measurement apparatus 100 is switched to document box mode. When the copier function is selected, the health data measurement apparatus 100 is switched to copy mode. When the printer function is selected, the health data measurement apparatus 100 is switched to printer mode. When the FAX function is selected, the image forming apparatus 10 is switched to FAX mode.

The network I/F 150 is an interface for performing data communication via the external network N1. The network I/F 150 and the near field communication circuit 120 are electrically connected to the ASIC 106 via the PCI bus 122.

The connection I/F 160 is an interface for performing USB communication with a USB device connected to the health data measurement apparatus 100 via a USB port.

The controller 110 receives a command issued via the operation panel 140 or a command issued by an external apparatus, and processes a job instructed by the command.

The following description will focus on configurations related to the engine controller 130, the scanner 131, and the printer 132, which relate to features of the present invention.

The printer 132 includes an image forming device 132a. The image forming device 132a includes components such as a photoconductor (i.e., a latent image bearer), an optical writing device, a developing device, a transfer device, a cleaning device, and a fixing device. The optical writing device forms an electrostatic latent image on the photoconductor. The developing device supplies toner to the electrostatic latent image on the photoconductor to visualize the electrostatic latent image into a toner image. The transfer device transfers the toner image on the photoconductor onto a recording medium transported to the transfer device. The cleaning device removes toner residue remaining on the photoconductor after the transfer process. The fixing device fixes the toner on the recording medium.

The developing device of a color image forming apparatus includes respective developing devices for different colors of toner used therein, e.g., a yellow (Y) developing device, a cyan (C) developing device, a magenta (M) developing device, and a black (K) developing device. The color image forming apparatus uses toner bottles each as a device for replenishing the corresponding developing device with toner of the corresponding color. The image forming device 132a further supplies infrared toner to the electrostatic latent image on the photoconductor to form a non-visualized toner image on the recording medium.

A hardware configuration of the health management server 16 will be described.

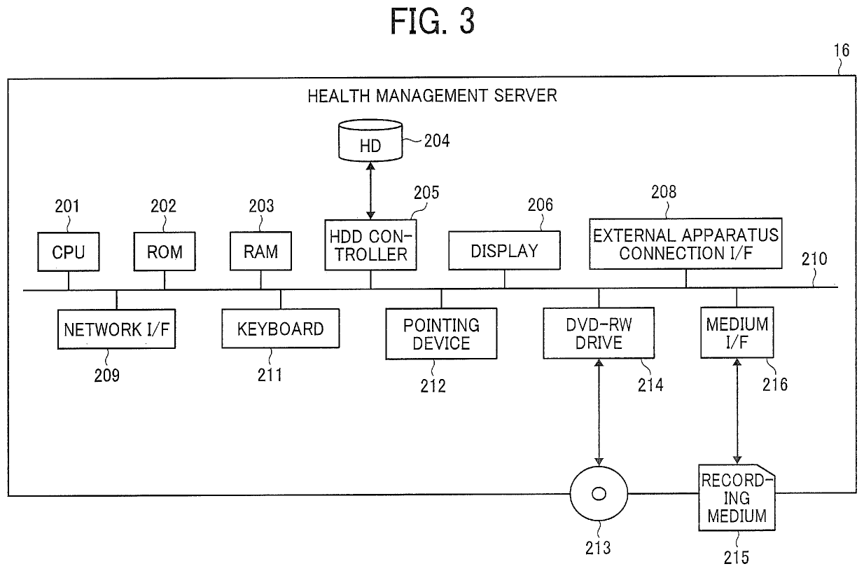

FIG. 3 is a diagram illustrating a hardware configuration of the health management server 16 illustrated in FIG. 1. The following description will be given of a hardware configuration of a PC implementing the health management server 16.

As illustrated in FIG. 3, the health management server 16 implemented by a computer includes a CPU 201, a ROM 202, a RAM 203, an HD 204, an HDD controller 205, a display 206, an external apparatus connection I/F 208, a network I/F 209, a bus line 210, a keyboard 211, a pointing device 212, a DVD-rewritable (DVD-RW) drive 214, and a medium I/F 216.

The CPU 201 controls overall operation of the PC. The ROM 202 stores a program used to drive the CPU 201, such as an initial program loader (IPL). The RAM 203 is used as a work area for the CPU 201. The HD 204 stores various data for programs, for example. The HDD controller 205 controls writing and reading of various data to and from the HD 204 under the control of the CPU 201.

The display 206 displays various information such as a cursor, menus, windows, text, and images. The external apparatus connection I/F 208 is an interface for connecting the health management server 16 to various external apparatuses. The external apparatuses in this case include a USB memory and a printer, for example. The network I/F 209 is an interface for performing data communication via a communication network N (e.g., the network N1, N2, or N3). The bus line 210 includes an address bus and a data bus for electrically connecting the CPU 201 and the other components in FIG. 3 to each other.

The keyboard 211 is an input device including a plurality of keys for inputting text, numerical values, and various instructions, for example. The pointing device 212 is an input device used to select and execute various instructions, select a processing target, and move the cursor, for example. The DVD-RW drive 214 controls writing and reading of various data to and from a DVD-RW 213 as an example of a removable recording medium. The removable recording medium is not limited to the DVD-RW, and may be a DVD-recordable (DVD-R), for example. The medium I/F 216 controls writing (i.e., storage) and reading of data to and from a recording medium 215 such as a flash memory.

A functional configuration of the health management system 1 will be described.

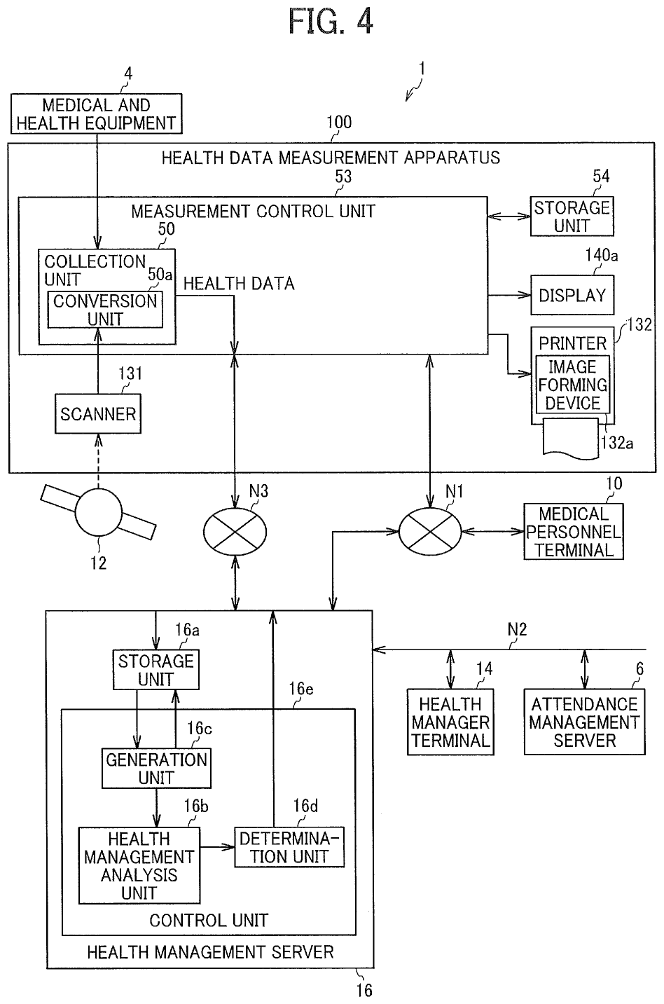

FIG. 4 is a block diagram illustrating a functional configuration of the health management system 1 of the first embodiment. The health management system 1 includes the medical and health equipment 4, the health data measurement apparatus 100, the smartwatch 12, the health management server 16, the medical personnel terminal 10, the health manager terminal 14, the attendance management server 6, and the networks N1, N2, and N3.

The medical and health equipment 4 measures the health data related to the body of the user 20. As described above, the medical and health equipment 4 is connected to devices such as the sphygmomanometer 4a, the body composition analyzer 4b, the blood sugar meter 4c, and the thermometer 4d, and the health data obtained from these devices of the medical and health equipment 4 is processed by the health data measurement apparatus 100.

The health data measurement apparatus 100 includes a measurement control unit 53 (i.e., first circuitry), a storage unit 54 (i.e., a second memory), the scanner 131, the display 140a (i.e., a display), and the printer 132.

The measurement control unit 53 includes a collection unit 50, which includes a conversion unit 50a. The collection unit 50 collects, from the medical and health equipment 4, the health data of the user 20 and items representing attributes of the health data.

When a display screen of the smartwatch 12 is placed on a reading surface of the scanner 131, the scanner 131 reads the two-dimensional code displayed on the smartwatch 12.

The conversion unit 50a converts the two-dimensional code read from the smartwatch 12 into the health data.

The display 140a displays a message prompting the user 20 to take a measurement of a particular item.

The measurement control unit 53 controls the display 140a to display a message prompting the user 20 to take a measurement of a required check item.

The storage unit 54 stores an operation manual P2 (see FIG. 1) of the medical and health equipment 4.

The printer 132 prints the operation manual P2 on a recording medium.

When a message is displayed on the display 140a, the measurement control unit 53 controls the display 140a to display the operation manual P2 or controls the printer 132 to print the operation manual P2.

The health management server 16 includes a storage unit 16a (i.e., a first memory) and a control unit 16e (i.e., second circuitry). The storage unit 16a stores the health data received from the health data measurement apparatus 100. The control unit 16e includes a generation unit 16c, a health management analysis unit 16b, and a determination unit 16d. These units implementing the respective functions in the control unit 16e may be disposed outside the control unit 16e.

Based on the health data and items received from the collection unit 50 of the health data measurement apparatus 100, the generation unit 16c generates the health management data representing the health state of the user 20.

The health management analysis unit 16b analyses the health management data read from the storage unit 16a.

In the health management server 16, at least one of an item indicating a worsening health state of the user 20, an item with insufficient measurement data, or an item lacking measurement data included in the health management data analyzed by the health management analysis unit 16b is determined as the required check item, and the thus-determined required check item is transmitted to the determination unit 16d as a determination result.

The health management analysis unit 16b calculates, for each of health management data items, the value of correlation (i.e., a first value of correlation) between the health management data read from the storage unit 16a and standard health management data. When the value of correlation meets a particular condition (i.e., a first particular condition), the health management analysis unit 16b extracts the required check item.

Based on the analysis by the health management analysis unit 16b, the determination unit 16d determines whether the health management data includes the required check item.

Based on the health management data generated by the generation unit 16c, the determination unit 16d determines whether the health management data includes the required check item, which is at least one of the item indicating a worsening health state of the user 20, the item with insufficient measurement data, or the item lacking measurement data.

If the determination unit 16d determines that the health management data includes the required check item, the control unit 16e transmits, to the health data measurement apparatus 100, a message prompting the user 20 to take a measurement of the required check item, and requests the health data measurement apparatus 100 to transmit the health data of the user 20 collected to the health data measurement apparatus 100 from the medical and health equipment 4.

The attendance management server 6 manages the working hours of the user 20.

The health management analysis unit 16b acquires the working hours data of the user from the attendance management server 6, and calculates, for each of health data items, the value of correlation (i.e., a second value of correlation) between the working hours data and the health data. Then, if the value of correlation meets a particular condition (i.e., a second particular condition), the health management analysis unit 16b extracts the required check item.

For example, the configuration disclosed in Japanese Unexamined Patent Application Publication No. 2017-107560 is applicable to the health management analysis unit 16b.

The health management system 1 further includes the medical personnel terminal 10 operated by the medical personnel 10a (see FIG. 1) who provide explanations of the medical and health equipment 4.

The health management system 1 further includes the smartwatch 12, the scanner 131, and the collection unit 50 including the conversion unit 50a.

The smartwatch 12 stores the health data of the user 20 and displays the stored health data as the two-dimensional code. The smartwatch 12 has functions of storing the health data of the user 20 (e.g., the blood pressure, the changes in the pulse, the amount of activity, and the length and depth of sleep) and displaying the stored health data as the two-dimensional code to be read by the scanner 131. The smartwatch 12 calculates the blood pressure and the pulse with sensors such as the optical sensor and the contact pressure sensor, and calculates the amount of activity with the acceleration sensor and the positional information of the user 20. Based on the statistical results obtained with these sensors, the smartwatch 12 further measures the length and depth of sleep.

The thermometer 4d measures the body temperature of the user 20 in a non-contact manner. In recent years, devices capable of measuring the body temperature in a non-contact manner, such as an infrared thermography, have been widely used. Such devices are used in fever check at airports, for example. With a non-contact type thermometer included in the health data measurement apparatus 100 as the thermometer 4d, body temperature information of the user 20 logged in to the health data measurement apparatus 100 is automatically acquired. The health data measurement apparatus 100 may, of course, include a typical contact-type thermometer as the thermometer 4d.

Further, a known technique may be employed as a method of measuring in real time the temperature of the skin of a human body (i.e., the body surface).

The measurement control unit 53 controls the display 140a to display a message prompting the user 20 to place the palm of a hand of the user 20 on the reading surface of the scanner 131, and issues a read command to the scanner 131. The measurement control unit 53 thereby acquires image data of the scanned palm of the hand of the user 20 from the scanner 131, and analyzes components contained in biological tissues of the palm of the hand of the user 20 based on the image data.

The scanner 131 includes a light emitting device and a light receiving device. Based on the difference between the spectrum of the light emitted from the light emitting device and the spectrum of the light reflected from the palm of the hand and received by the light receiving device, the contained components are identified to make a diagnosis such as insufficient vegetable intake.

For example, the configurations disclosed in Japanese Translations of PCT International Application Publication Nos. JP-T-2011-523059 and JP-T-2012-515349 are applicable to the method of measuring data from the palm of a hand to make a diagnosis such as insufficient vegetable intake.

The health management system 1 further includes the printer 132 including the image forming device 132a. The image forming device 132a supplies infrared toner to the electrostatic latent image on the photoconductor to form a non-visualized toner image on a recording medium.

The health manager terminal 14 is a PC operated by the health manager. If the health data of the required check item is not acquired despite the message prompting the user 20 to take a measurement of the required check item, the control unit 16e of the health management server 16 transmits to the health manager terminal 14 an electronic mail (email) including a message notifying that the health data of the required check item is missing. The health management server 16 included in the health management system 1 acquires the health data of the user 20 and manages the health data in association with the corresponding user ID (i.e., employee ID). The health management server 16 analyses the stored health data of the individual user 20. Then, if recent information of the health data is missing, the health management server 16 issues a data request to request the health data.

The health manager takes measures in response to the notification. If the user 20 has not taken a measurement of the required check item, the health manager urges the user 20 to take the measurement. The health manager has the authority to view the health data of the individual user 20 (i.e., employee), and thus is capable of constantly monitoring the health state of the employee and promoting the health maintenance of the employee.

An operation of the health management system 1 will be described.

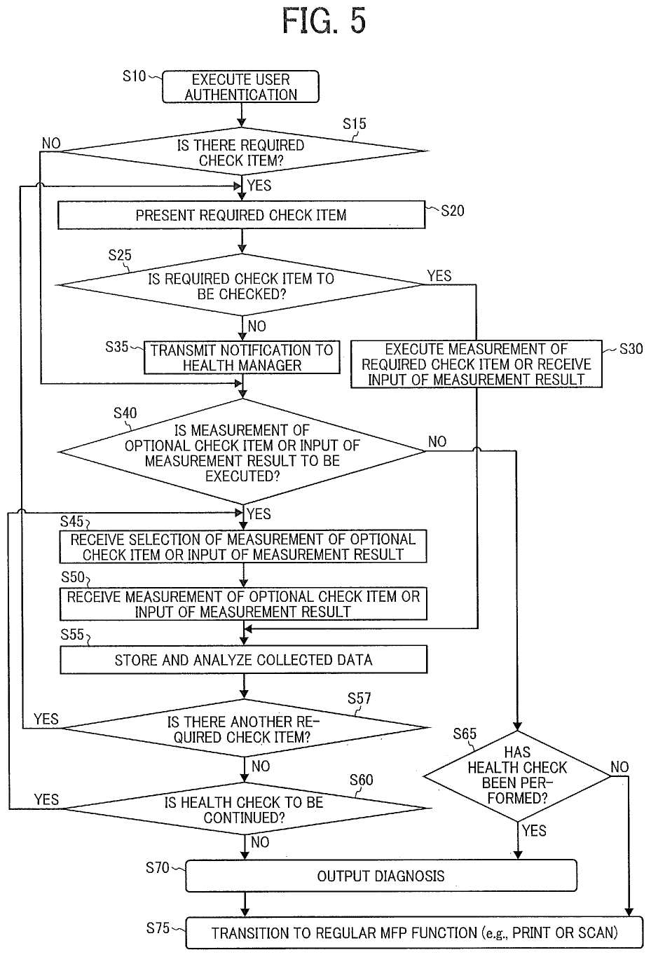

FIG. 5 is a flowchart illustrating an operation of the health management system 1 of the first embodiment. When the user 20 holds the employee ID card 22 thereof over the card reader 2, the card reader 2 transmits the employee ID read from the employee ID card 22 to the health management server 16 via the health data measurement apparatus 100. Then, user authentication by the health management server 16 is executed.

At step S10, the control unit 16e executes the user authentication. When the user 20 holds the employee ID card 22 thereof over the card reader 2, the measurement control unit 53 reads the employee ID from the employee ID card 22 via the card reader 2 and transmits the read employee ID to the health management server 16.

The control unit 16e executes the authentication process by checking whether the read employee ID is registered in the storage unit 16a. Then, if it is determined that the user is a valid employee, the control unit 16e allows the user 20 to log in to the health management system 1. Alternatively, the user 20 may be asked to input a password via the operation device 140b before the authentication process, and the password may be transmitted to the health management server 16 to check whether the combination of the employee ID and the password is registered in the storage unit 16a.

With the completion of the authentication process, the health data measurement apparatus 100 and the health management server 16 are communicably connected to each other.

At step S15, the control unit 16e determines, with the determination unit 16d, whether there is a required check item. That is, the determination unit 16d determines whether the health management data of the user 20 includes the required check item, which is at least one of the item indicating a worsening health state of the user 20, the item with insufficient measurement data, or an item lacking measurement data.

The health management data of the user 20 is stored in the storage unit 16a. The determination unit 16d therefore reads from the storage unit 16a the health management data associated with the employee ID of the user 20.

The health management data may be configured as follows:

TABLE 1

| | | | | |
|---|---|---|---|---|
| | Measurement | Numerical | Standard | Evaluation |
| Item | date | value | value | level |

Blood pressure
Body weight
Body fat percentage
Blood sugar level
Pulse
Amount of exercise
Amount of sleep
Body temperature

As illustrated in TABLE 1, the health management data includes columns of item, measurement date, numerical value, standard value, and evaluation level and rows of blood pressure, body weight, body fat percentage, blood sugar level, pulse, amount of exercise, amount of sleep, and body temperature.

With reference to TABLE 1, the determination unit 16d determines the item lacking the information of recent measurement by the user 20, the item indicating a worsening health state of the user 20, the item with insufficient measurement data, or the item lacking measurement data as the required check item. At step S20, the control unit 16e presents the user 20 with a message prompting the user 20 to take a measurement of the required check item. If it is determined at step S15 that there is no required check item (NO at step S15), the control unit 16e proceeds to step S40 to execute the measurement of an optional check item. The user 20 has previously been notified of the required check item via email. At step S20, the control unit 16e transmits the required check item to the health data measurement apparatus 100 and controls the display 140a to present (i.e., display) the required check item. That is, the control unit 16e transmits to the health data measurement apparatus 100 the message prompting the user 20 to take a measurement of the required check item determined by the determination unit 16d and controls the display 140a to display the message.

At step S25, the control unit 16e allows the user 20 to decide whether to check the required check item determined by the determination unit 16d. If the user 20 decides to check the required check item, the control unit 16e proceeds to step S30 to execute the measurement. If the user 20 decides not to check the required check item, the control unit 16e proceeds to step S35 to transmit an email to the health manager terminal 14 to notify the health manager of the decision of the user 20.

At step S30, the control unit 16e remote controls the health data measurement apparatus 100 to execute the measurement of the required check item.

A method of using the medical and health equipment 4 with the display 140a will be described below. Alternatively, however, print data describing the method of using the medical and health equipment 4 may be output to the printer 132 to print the print data on a recording medium (e.g., recording sheet).

The health data measurement apparatus 100 is connected to the medical personnel terminal 10 via the external network N1. If having any question, therefore, the user 20 is able to receive follow-up assistance from a nurse or doctor via video call with the display 140a.

The health data measurement apparatus 100 is also capable of reading a medical examination report P1 (see FIG. 1) via the scanner 131 and collecting the health data from the medical examination report P1 through a character recognition process. The health data read from the medical examination report P1 is transmitted to and stored in the health management server 16 on a network N3, enabling observation over time of the health state of the user 20.

If the required check item is the body composition, the user 20 measures, for example, the body weight and the body fat percentage of the user 20 with the body composition analyzer 4b included in the medical and health equipment 4. The collection unit 50 collects the health data and item names of the body weight and the body fat percentage of the user 20 from the body composition analyzer 4b. The collection unit 50 temporarily stores the collected health data and item names in the storage unit 54. The collection unit 50 further reads the health data and item names from the storage unit 54, and transmits the read health data and item names to the health management server 16 to store the health data and item names in the storage unit 16a.

Based on the health data and item names stored in the storage unit 16a, the generation unit 16c generates the health management data representing the health state of the user 20.

The determination unit 16d writes the health management data generated by the generation unit 16c to the storage unit 16a to store the health management data in the storage unit 16a. Consequently, the health management data is generated which includes the measurement dates and the numerical values corresponding to the item names of the measured items and general standard values of the items, as illustrated in TABLE 1.

If a button for specifying the decision of the user 20 not to take a measurement of the required check item is selected on the display 140a of the health data measurement apparatus 100 (NO at step S25), the control unit 16e transmits an email to the health manager terminal 14 at step S35 to notify the health manager of the decision of the user 20. Herein, the health manager is assumed to be a supervisor of the user 20 or an occupational physician.

The health manager operates the health manager terminal 14 to display, on the display 140a, a message to the user 20 who has not taken a measurement of the required check item, i.e., a message prompting the user 20 to take a measurement of the required check item. For instance, if the user 20 does not have enough time to take a measurement of the required check item due to a high workload, the health manager makes arrangements to reduce the workload of the user 20 to secure sufficient time for the user 20 to take the measurement, for example.

At step S40 subsequent to the processes of steps S15 to S35, the control unit 16e is ready to execute the measurement of the optional check item. Herein, the control unit 16e allows the user 20 to decide whether to take a measurement of the optional check item or let the scanner 131 read the health data.

The control unit 16e controls the display 140a to display a message prompting the user 20 to select a button for specifying the measurement of the optional check item or the input of the measurement result. Then, when an item displayed on the display 140a is pressed by the user 20, the control unit 16e acquires the pressed item at step S45.

At step S50, the control unit 16e receives the measurement of the optional check item or the input of the measurement result. Consequently, the health management data is generated which includes the measurement dates and the numerical values corresponding to the item names of the measured items and the general standard values of the items, as illustrated in TABLE 1.

If the optional check item is the blood pressure, the user 20 measures the blood pressure with the sphygmomanometer 4a included in the medical and health equipment 4. The collection unit 50 collects the health data and the item name of the blood pressure of the user 20 from the sphygmomanometer 4a.

Based on the health data and the item name collected by the collection unit 50 of the health data measurement apparatus 100 and stored in the storage unit 16a, the generation unit 16c generates the health management data representing the health state of the user 20.

The control unit 16e writes the health management data generated by the generation unit 16c to the storage unit 16a to store the health management data in the storage unit 16a.

At step S55, the control unit 16e receives from the health data measurement apparatus 100 the health data obtained through the measurement of the optional check item or through the input of the measurement result, and stores the received health data in the storage unit 16a. Specifically, the health data measurement apparatus 100 temporarily stores the health data in the storage unit 54, and transmits the health data to the health management server 16 to store the health data in the storage unit 16a. In this process, the measurement result obtained with the health data measurement apparatus 100 or the health data input via the scanner 131 is stored in the storage unit 54.

Further, at step S55, the control unit 16e receives the health data obtained by the health data measurement apparatus 100 through the measurement of the required check item or through the input of the measurement result, and stores the received health data in the storage unit 16a. In this process, the measurement result obtained by the health data measurement apparatus 100 or the health data input via the scanner 131 (hereinafter collectively referred to as the health data) is temporarily stored in the storage unit 54. The health data is then transmitted to the health management server 16 by the health data measurement apparatus 100 to be stored in the storage unit 16a. Based on the health data and item names stored in the storage unit 16a, the generation unit 16c generates the health management data representing the health state of the user 20. The control unit 16e writes the health management data generated by the generation unit 16c to the storage unit 16a to store the health management data in the storage unit 16a. Consequently, the storage unit 16a stores the health management data including the measurement dates and the numerical values corresponding to the item names of the measured items and the general standard values of the items, as illustrated in TABLE 1.

At step S55, the control unit 16e then analyzes the health management data of the user 20. In the health management server 16, the storage unit 16a stores the health management data. The health management analysis unit 16b analyzes the health management data read from the storage unit 16a. In the health management data analyzed by the health management analysis unit 16b, at least one of the item indicating a worsening health state of the user 20, the item with insufficient measurement data, or the item lacking measurement data is determined as the required check item and transmitted to the determination unit 16d.

The health management analysis unit 16b analyzes, for each of health management data items, whether the read health management data is deviated from a standard value, and evaluates the health management data of the item on a scale of 1 to 5.

The health management analysis unit 16b further calculates, for each of health management data items, the value of correlation between the health management data of the user 20 and standard health management data. Then, if the value of correlation meets a particular condition, the health management analysis unit 16b determines a worsening health state of the user 20, and evaluates the health management data of the item on the scale of 1 to 5.

In the present example, when the obtained measurement result is deviated from the standard value, the health management analysis unit 16b determines that the measurement result indicates a worsening health state of the user 20.

TABLE 2 given below presents an example of 5-point scale evaluation used in municipal medical examination, which is applicable to the present embodiment.

TABLE 2

| Evaluation | | |
|---|---|---|
| level | Determination | Description |
| 1 | Normal | No anomalies detected by examination, or |
| | | slight anomalies detected but currently not |
| | | considered pathological. No interference |
| | | with daily life. |
| 2 | Caution | Mild anomalies detected. Pay attention to |
| | required | daily life and keep monitoring the condition |
| | | until next medical examination. Should you |
| | | have any concern or symptom, consult your |
| | | doctor. |
| 3 | Observation | Anomalies detected. Continuous observation |
| | required | required. Re-examination in 3 to 6 months |
| | | recommended even with no symptoms |
| | | noticed. |
| 4 | Under | Continue treatment following instruction |
| | treatment | from your family doctor. |
| 5 | Detailed | Anomalies detected. Detailed examination |
| | examination | required. Make sure to consult a doctor. |
| | required | |

If the date of registration of the measurement date of an item of the health management data of the user 20 is over a year ago from the current date, for example, the health management analysis unit 16b determines the item as the item with insufficient measurement data.

If the data of the measurement date and the numerical value corresponding to an item of the health management data of the user 20 is not registered, the health management analysis unit 16b determines the item as the item lacking measurement data.

The health management analysis unit 16b may thus determine whether the health management data of the user 20 includes the required check item by determining at least one of the item indicating a worsening health state of the user 20, the item with insufficient measurement data, or the item lacking measurement data as the required check item.

Alternatively, the determination unit 16d may determine whether the health management data of the user 20 includes the required check item by determining at least one of the item indicating a worsening health state of the user 20, the item with insufficient measurement data, or the item lacking measurement data as the required check item.

The health management analysis unit 16b further calculates, for each of health management data items, the value of correlation between the health management data read from the storage unit 16a and the standard health management data, and extracts the required check item when the value of correlation meets the particular condition.

The attendance management server 6 manages the working hours of the user 20. The control unit 16e acquires the working hours data of the user 20 from the attendance management server 6, and calculates, for each of health data items, the value of correlation between the working hours data and the health data. Then, when the value of correlation meets a particular condition, the control unit 16e extracts the required check item.

At step S57, the control unit 16e determines whether there is another required check item. If it is determined that there is another required check item (YES at step S57), the control unit 16e automatically returns to step S20 to check the another required check item. If it is determined that there is not another required check item (NO at step S57), the control unit 16e automatically proceeds to step S60.

At step S60, there is no more required check item. If the user 20 wants to additionally take a measurement of the optional check item, i.e., to continue health check (YES at step S60), the control unit 16e returns to step S45. If the user 20 decides not to additionally take a measurement of the optional check item, i.e., not to continue the health check (NO at step S60), the control unit 16e proceeds to step S70 to output the diagnosis. The decision of whether to additionally take a measurement of the optional check item is made by the user 20.

At step S65, the control unit 16e determines whether the measurement of the item or the input of the measurement result has been executed (i.e., whether the health check has been executed) at least once before this step. If it is determined that the measurement of the item or the input of the measurement result has been executed at least once, the control unit 16e proceeds to step S70 to output the diagnosis. If it is determined that the measurement of the item or the input of the measurement result has not been executed once, the control unit 16e proceeds to step S75. The determination at this step is automatically made by the health data measurement apparatus 100.

At step S70, the control unit 16e outputs the diagnosis. The diagnosis includes the current measurement result, the changes in the measurement result, and comments (e.g., “No anomalies are detected,” “XXX is worsening and caution is required,” or “Consultation with a doctor about XXX is required.”).

At step S75, the health data measurement apparatus 100 transitions to the regular function of the MFP.

A series of user interfaces (UIs) displayed on the health data measurement apparatus 100 in the first embodiment will be described.

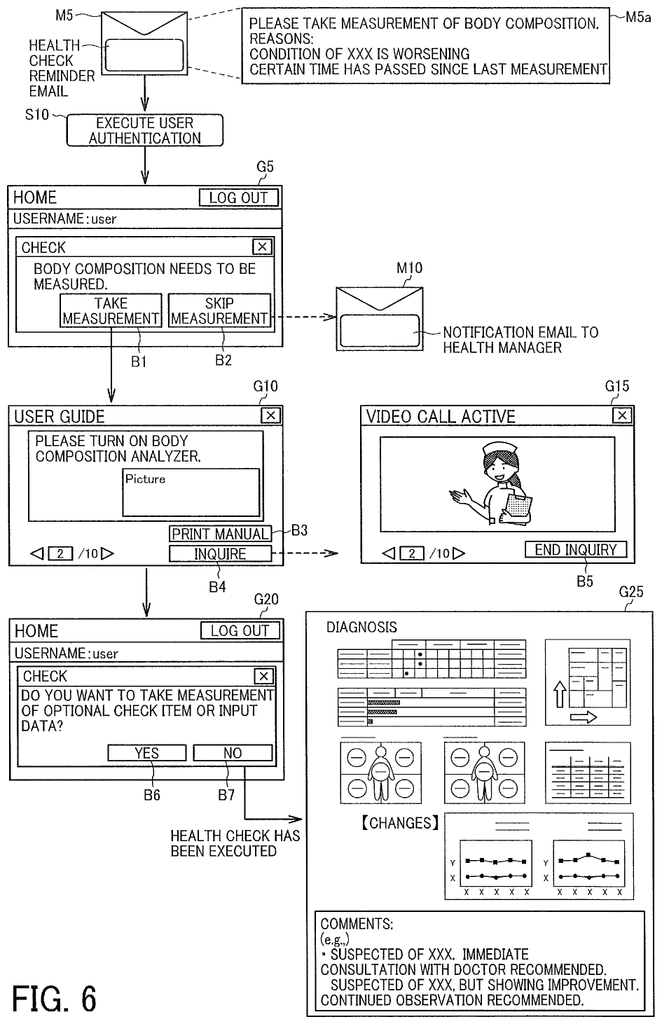

FIG. 6 is a diagram illustrating a series of UIs displayed on a front surface of the health data measurement apparatus 100 in the first embodiment to prompt the user 20 to take a measurement of the body composition based on a determination that the measurement by the user 20 is necessary, and guide the user 20 to complete the measurement.

The health management server 16 analyses the stored health information of the individual user 20, and thereby requests the health data measurement apparatus 100 to execute the measurement of the body composition, for example, as the required check item. If a change over time of the body composition and a possible disease associated therewith are predicted, and if there is an item the measurement of which has not been taken for a certain period of time, the health management server 16 requests the health data measurement apparatus 100 to execute the measurement of the body composition to accurately examine the change over time.

The user 20 checks the contents of an email M5 and approaches the health data measurement apparatus 100. After the user 20 succeeds in user authentication with the health data measurement apparatus 100, the health data measurement apparatus 100 and the health management server 16 are communicably connected to each other.

At step S10, the control unit 16e controls the display 140a to display a screen G5 to prompt the user 20 to take a measurement of the body composition.

At step S25, if a “SKIP MEASUREMENT” button B2 is pressed through user operation to avoid the measurement, the control unit 16e transmits an email M10 to the health manager terminal 14 at step S35. Further, the user 20 is restricted in using the health data measurement apparatus 100.

If a “TAKE MEASUREMENT” button B1 is pressed through user operation to take the measurement, on the other hand, the control unit 16e controls the display 140a to display a screen G10 at step S30 to display a guide on the use of the body composition analyzer 4b. If a “PRINT MANUAL” button B3 is pressed through user operation, the explanation of the operation manual P2 is printed on a recording medium (i.e., recording sheet). If the user 20 has a question, the user 20 may press an “INQUIRE” button B4 to display a screen G15. Thereby, the health data measurement apparatus 100 is connected to the medical personnel terminal 10 via the external network N1, enabling the user 20 to directly receive explanations from a nurse. Consequently, the user 20 is able to measure the body composition with the body composition analyzer 4b while receiving explanations from the health data measurement apparatus 100 and the nurse.

At step S40, the control unit 16e controls the display 140a to display a screen G20 displaying a message asking the user 20 whether to take a measurement of the optional check item or input the data of the measurement result. The screen G20 displays a “YES” button B6 and a “NO” button B7. If the “YES” button B6 is pressed through user operation, the measurement of the optional check item is executed. If YES at step S65, the control unit 16e controls the display 140a to display a screen G25 displaying the diagnosis, enabling the user to check the health data.

A series of UIs displayed on the health data measurement apparatus 100 in a second embodiment of the present invention will be described.

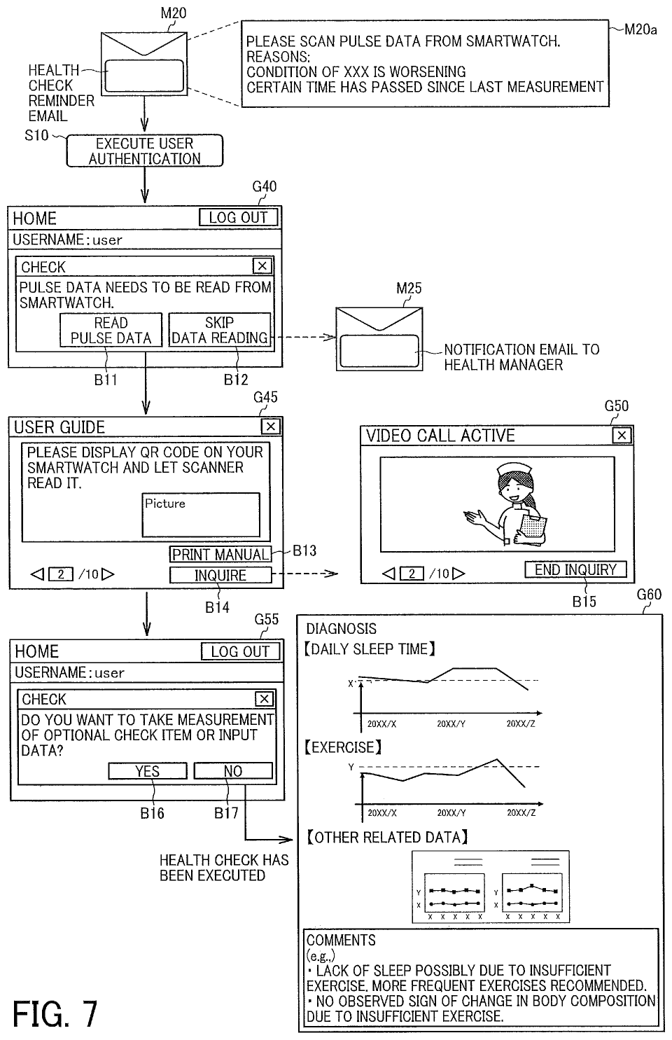

FIG. 7 is a diagram illustrating a series of UIs displayed on the front surface of the health data measurement apparatus 100 in the second embodiment to notify the user 20 of a determination that the measurement of an item measurable by the user 20 with the smartwatch 12 is necessary and guide the user 20 to complete data reading.

The health management server 16 analyzes the stored health information of the individual user 20. If recent information of the health data of the user 20 is missing, the health management server 16 issues a data request to the health data measurement apparatus 100 to request the health data measurement apparatus 100 to transmit the health data stored in the smartwatch 12 to the health management server 16. In the present example, when recent information of pulse data is missing, the health management server 16 issues the data request to acquire the recent information of the pulse data from the smartwatch 12.

The user 20 checks an email M20 and approaches the health data measurement apparatus 100.

At step S10, after the control unit 16e executes the user authentication, the health data measurement apparatus 100 and the health management server 16 are communicably connected to each other. In this state, the health management server 16 transmits a message to the health data measurement apparatus 100 and controls the display 140a of the health data measurement apparatus 100 to display a screen G40 with the message, which reads “PULSE DATA NEEDS TO BE READ FROM SMARTWATCH.”

At step S25, if a “SKIP DATA READING” button B12 is pressed through user operation at to avoid the measurement, the control unit 16e transmits an email M25 to the health manager (i.e., to the health manager terminal 14) at step S35. Further, the user 20 is restricted in using the health data measurement apparatus 100.

If a “READ PULSE DATA” button B11 is pressed through user operation to take the measurement, on the other hand, the control unit 16e controls the display 140a to display a screen G45 at step S30 to display a data reading method using the smartwatch 12. That is, if the “READ PULSE DATA” button B11 is pressed through user operation to take the measurement, the control unit 16e controls the display 140a to display the screen G45 at step S30 to display a use guidance message “PLEASE DISPLAY QR CODE ON YOUR SMARTWATCH AND LET SCANNER READ IT.” QR code (registered trademark) represents quick response code.

If a “PRINT MANUAL” button B13 is pressed through user operation in this case, the explanation of the operation manual P2 is printed on a recording medium (i.e., recording sheet). If the user 20 has a question, the user 20 may press an “INQUIRE” button B14 to display a screen G50. Thereby, the health data measurement apparatus 100 is connected to the medical personnel terminal 10 via the external network N1, enabling the user 20 to directly receive explanations from a nurse. Consequently, the user 20 is able to perform the pulse data reading with the smartwatch 12 while receiving explanations from the health data measurement apparatus 100 and the nurse.

At step S40, the control unit 16e controls the display 140a to display a screen G55 displaying a message asking the user 20 whether to take a measurement of the optional check item or input the data of the measurement result. The screen G55 displays a “YES” button B16 and a “NO” button B17. If the “YES” button B16 is pressed through user operation, the measurement of the optional check item is executed.

If YES at step S65, the control unit 16e controls the display 140a to display a screen G60 displaying the diagnosis, enabling the user 20 to check the health data.

A series of UIs displayed on the health data measurement apparatus 100 in a third embodiment of the present invention will be described.

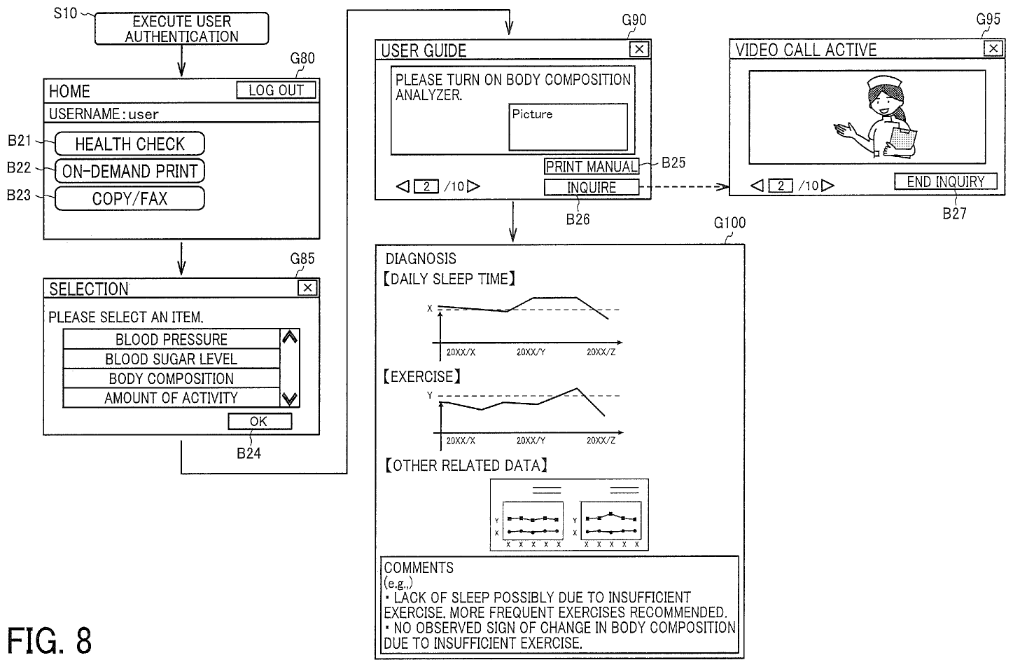

FIG. 8 is a diagram illustrating a series of UIs displayed on the front surface of the health data measurement apparatus 100 in the third embodiment to guide the user 20 to use the health data measurement apparatus 100 when there is no item for which the measurement or data reading is necessary.

The user 20 first approaches the health data measurement apparatus 100. After the user authentication is executed with the health data measurement apparatus 100 at step S10, the health data measurement apparatus 100 and the health management server 16 are communicably connected to each other. In this state, the control unit 16e controls the display 140a to display a screen G80 including a “HEALTH CHECK” button B21, an “ON-DEMAND PRINT” button B22, and a “COPY/FAX” button B23. If the “HEALTH CHECK” button B21 is pressed in this case, the screen G80 transitions to a screen G85.

At step S45, the control unit 16e receives user selection of one of a plurality of health check items displayed on the screen G85. The screen G85 displays items such as blood pressure, blood sugar level, body composition, and amount of activity, for example, as the plurality of health check items. When the user 20 presses (i.e., selects) one of the items and presses an “OK” button B24, the screen G85 transitions to a screen G90.

The following description will be given on the assumption that the item “body composition” has been selected.

As illustrated in FIG. 8, the display 140a displays the screen G90 displaying a message “PLEASE TURN ON BODY COMPOSITION ANALYZER” as a user guide of the body composition analyzer 4b. The screen G90 further displays a “PRINT MANUAL” button B25 and an “INQUIRE” button B26.

In this case, the “PRINT MANUAL” button B25 may be pressed through user operation to print the explanation of the operation manual P2 on a recording medium (i.e., recording sheet). Further, if the user 20 has a question, the “INQUIRE” button B26 may be pressed through user operation to display a screen G95. Thereby, the health data measurement apparatus 100 is connected to the medical personnel terminal 10 via the external network N1, enabling the user 20 to directly receive explanations from a nurse. Consequently, the user 20 is able to measure the body composition with the body composition analyzer 4b while receiving explanations from the health data measurement apparatus 100 and the nurse.

At step S70, the control unit 16e outputs the diagnosis. A screen G100 displaying the current measurement result is displayed as the diagnosis.

According to a typical health management system, the measurement of the health data is voluntary. Such a system may fail to execute the measurement of an item indicating a worsening health state of a user, resulting in the deterioration of the condition of the user related to the missed measurement item.

According to the present embodiments, on the other hand, the display 140a displays the message prompting the user 20 to take a measurement of the required check item of the health management data related to the body of the user 20. That is, at least one of the item indicating a worsening health state of the user 20, the item with insufficient measurement data, or the item lacking measurement data is determined as the required check item, and the message prompting the user 20 to take a measurement of the required check item is displayed on the display 140a. Thereby, the user 20 is guided to appropriately take a measurement of the health data item relevant to the health maintenance of the body of the user 20.

The MFP used as the health data measurement apparatus 100 has been widely used. Further, the health management server 16 is pre-installed with the function of communicating with another device or apparatus via a network. The health management server 16 therefore performs various operations via the network, such as collecting information of how the printer function has been used by the user 20 and logs of the user 20, transmitting notification of consumable replacement time, changing parameters of equipment, and updating software.

With the above-described communication of the health management server 16, the health management server 16 is capable of collecting in bulk big data related to the health management. With the use of the thus-collected data, a health management section of the company or an external medical institute is able to obtain various useful information such as a sudden anomaly in an individual, the characteristics of the respective sections of the company, and a local epidemic.

Further, since the MFP used as the health data measurement apparatus 100 has been widely used, the health data collected by a plurality of health data measurement apparatuses 100 may be collected in bulk in the health management server 16. With the use of the data stored in the health management server 16, the health management section of the company or the external medical institute is able to obtain various useful information such as a sudden anomaly occurring in the body of an individual (e.g., attack of fever), the characteristics of the respective sections of the company, and a local epidemic. For example, an anomaly may be detected in the early stages based on an increase in the number of users suffering from fever.

With the acquired health data thus stored in bulk in a database, the managerial or related party is able to check the stored information as necessary.

The health management system 1 according to at least one of the embodiments includes the health data measurement apparatus 100 and the health management server 16. The health data measurement apparatus 100 includes the medical and health equipment 4 capable of measuring the health data related to the body of the user 20 using the medical and health equipment 4. The health management server 16 is communicable with the health data measurement apparatus 100. The health data measurement apparatus 100 further includes the collection unit 50 (i.e., the first circuitry) that collects the health data of the user 20 from the medical and health equipment 4. The health management server 16 includes the storage unit 16a (i.e., the first memory) and the control unit 16e (i.e., the second circuitry) including the generation unit 16c, the health management analysis unit 16b, and the determination unit 16d. The storage unit 16a stores the health data of the user 20 from the health data measurement apparatus 100. Based on the health data of the user 20 stored in the storage unit 16a, the generation unit 16c generates the health management data representing the health state of the user 20. Based on the health management data generated by the generation unit 16c, the health management analysis unit 16b analyzes at least one of the item indicating a worsening health state of the user 20, the item the measurement with insufficient measurement data, or the item lacking measurement data as the required check item. Based on the analysis of the health management analysis unit 16b, the determination unit 16d determines whether the health management data includes the required check item. When it is determined by the determination unit 16d that the health management data includes the required check item, the control unit 16e transmits, to the health data measurement apparatus 100, the message prompting the user 20 to take a measurement of the required check item, and requests the health data measurement apparatus 100 to transmit the health data of the user 20 to the health management server 16.

According to the embodiment, when the determination unit 16d determines that the health management data includes the required check item, the message prompting the user 20 to take a measurement of the required check item is transmitted to the health data measurement apparatus 100 to be displayed. Further, with the health management server 16 storing in bulk the health data acquired from the health data measurement apparatus 100, the managerial or related party is able to check the stored information as necessary.

Thereby, the user 20 is guided to appropriately take a measurement of the health data item relevant to the health maintenance of the body of the user 20. Further, with the acquired health data stored in bulk, the managerial or related party is able to check the stored information as necessary.

The health data measurement apparatus 100 of the embodiment further includes the display 140a (i.e., the display). In response to receipt of a request from the health management server 16, the measurement control unit 53 controls the display 140a to display the message prompting the user 20 to take a measurement of the required check item, and transmits the health data of the user 20 collected from the medical and health equipment 4 to the health management server 16.

According to the embodiment, the message prompting the user 20 to take a measurement of the required check item is displayed, and the health data of the user 20 collected to the collection unit 50 from the medical and health equipment 4 is transmitted to the health management server 16. Thereby, the user 20 is guided to appropriately take a measurement of the health data item relevant to the health maintenance of the body of the user 20.

The health data measurement apparatus 100 of the embodiment further includes the storage unit 54 (i.e., the second memory) that stores the operation manual P2 of the medical and health equipment 4. When the message is displayed on the display 140a, the measurement control unit 53 controls the display 140a to display the operation manual P2.

According to the embodiment, when the message is displayed on the display 140a, the operation manual P2 is displayed on the display 140a.

The health data measurement apparatus 100 of the embodiment further includes the printer 132 and the storage unit 54 (i.e., the second memory) that stores the operation manual P2 of the medical and health equipment 4. When the message is displayed on the display 140a, the measurement control unit 53 controls the printer 132 to print the operation manual P2.

According to the embodiment, when the message is displayed on the display 140a, the operation manual P2 is printed by the printer 132.

The health management analysis unit 16b of the embodiment calculates, for each health management data item, the value of correlation (i.e., the first value of correlation) between the generated health management data and the standard health management data. Then, when the value of correlation meets a particular condition (i.e., the first particular condition), the health management analysis unit 16b extracts the required check item.

According to the embodiment, the health management analysis unit 16b calculates, for each health management data item, the value of correlation between the generated health management data and the standard health management data, and extracts the required check item when the value of correlation meets the particular condition.

The health management system 1 of the embodiment further includes the attendance management server 6 that manages the working hours of the user 20. The health management analysis unit 16b acquires the working hours data of the user 20 from the attendance management server 6, and calculates, for each health data item, the value of correlation (i.e., the second value of correlation) between the working hours data and the health data. Then, when the value of correlation meets a particular condition (i.e., the second particular condition), the health management analysis unit 16b extracts the required check item.

According to the embodiment, the health management analysis unit 16b acquires the working hours data of the user 20 from the attendance management server 6, calculates, for each health data item, the value of correlation between the working hours data and the health data, and extracts the required check item when the value of correlation meets the particular condition.

The health management system 1 of the embodiment further includes the medical personnel terminal 10 (i.e., the first terminal) operated by the medical personnel 10a who provide explanations of the medical and health equipment 4.

According to the embodiment, with the medical personnel terminal 10 operated by the medical personnel 10a who provide explanations of the medical and health equipment 4, the health management system 1 enables the medical personnel 10a to provide the explanations of the medical and health equipment 4.

The health data measurement apparatus 100 of the embodiment displays the display screen displaying the health data as the two-dimensional code. The health data measurement apparatus 100 further includes the scanner 131 that reads the displayed two-dimensional code. When the display screen of the smartwatch 12 displaying the health data as the two-dimensional code is placed on the scanner 131, the scanner 131 reads the two-dimensional code displayed on the smartwatch 12. The collection unit 50 includes the conversion unit 50a that converts the two-dimensional code read from the smartwatch 12 by the scanner 131 into the health data.

According to the embodiment, with the conversion unit 50a, the collection unit 50 converts the two-dimensional code read from the smartwatch 12 by the scanner 131 into the health data, and thus is able to collect the health data from the smartwatch 12.

The measurement control unit 53 of the embodiment controls the display 140a to display the message prompting the user 20 to place the palm of a hand of the user 20 on the reading surface of the scanner 131, and issues the read command to the scanner 131 to scan the palm of the hand of the user 20. Thereby, the measurement control unit 53 acquires the image data of the scanned palm of the hand of the user 20 from the scanner 131, and analyzes, based on the image data, the components contained in the biological tissues of the palm of the hand of the user 20.

According to the embodiment, the image data of the scanned palm of the hand of the user 20 is acquired from the scanner 131, and the components contained in the biological tissues of the palm of the hand of the user 20 are analyzed based on the image data.

The printer 132 of the embodiment includes the image forming device 132a that supplies the infrared toner to the electrostatic latent image on the photoconductor to form the non-visualized toner image on the recording medium.

According to the embodiment, the printer 132 supplies the infrared toner to the electrostatic latent image on the photoconductor and forms the non-visualized toner image on the recording medium with the image forming device 132a. Consequently, the toner image formed on the recording medium is made invisible to the third party, improving privacy protection.

The health management system 1 of the embodiment further includes the health manager terminal 14 (i.e., the second terminal) operated by the health manager. When the measurement control unit 53 fails to acquire the health data of the required check item corresponding to the message prompting the user 20 to take the measurement, the measurement control unit 53 transmits a message to the health manager terminal 14 to notify the lack of the health data of the required check item.

According to the embodiment, when the health data of the required check item is not acquired despite the message prompting the user 20 to take a measurement of the required check item, the measurement control unit 53 transmits a message to the health manager terminal 14 to notify the lack of the health data of the required check item, enabling the health manager to urge the user 20 corresponding to the lacked health data to take the measurement.

A program according to at least one of the embodiments is executed by the control unit 16e included in the health management server 16 of the above-described health management system 1. The control unit 16e is implemented by a processor. According to the embodiment, the functions of the control unit 16e are executed by the processor.

A health management method according to at least one of the embodiments is performed by the health management system 1 that includes the health data measurement apparatus 100 and the health management server 16. The health data measurement apparatus 100 includes the medical and health equipment 4 capable of measuring the health data related to the body of the user 20. The health management server 16 is communicable with the health data measurement apparatus 100.

The health management method includes storing the health data of the user 20 in the storage unit 16a (i.e., a memory) from the health data measurement apparatus 100. The health data of the user 20 is collected to the health data measurement apparatus 100 from the medical and health equipment 4. The health management method further includes generating, based on the health data of the user 20 stored in the storage unit 16a, the health management data representing the health state of the user 20, analyzing, based on the generated health management data, at least one of the item indicating a worsening health state of the user 20, the item with insufficient measurement data, or the item lacking measurement data as the required check item, determining, based on the analysis, whether the health management data includes the required check item, and based on a determination that the health management data includes the required check item, transmitting to the health data measurement apparatus 100 a message prompting the user 20 to take a measurement of the required check item, and requesting the health data measurement apparatus 100 to transmit the health data of the user 20.

According to the embodiment, when it is determined by the determining that the health management data includes the required check item, the message prompting the user 20 to take a measurement of the required check item is transmitted to the health data measurement apparatus 100 to be displayed. Further, the acquired health data is stored in bulk, enabling the managerial or related party to check the stored information as necessary.

Thereby, the user 20 is guided to appropriately take a measurement of the health data item relevant to the health maintenance of the body of the user 20. Further, with the acquired health data stored in bulk, the managerial or related party is able to check the stored information as necessary.

The health data measurement apparatus 100 according to at least one of the embodiments is connected to the health management server 16 via a network. The health data measurement apparatus 100 transmits the health data of the user 20 to the health management server 16 to store the health data in the health management server 16. The health data measurement apparatus 100 includes the display 140a (i.e., a display), the medical and health equipment 4, and the measurement control unit 53 (i.e., circuitry). The medical and health equipment 4 measures the health data related the body of the user 20. The measurement control unit 53 controls the measurement of the medical and health equipment 4. The collection unit 50 of the measurement control unit 53 collects the health data of the user 20 using the medical and health equipment 4. In response to receipt of a request from the health management server 16, the measurement control unit 53 controls the display 140a to display the message prompting the user 20 to take a measurement of the required check item, and transmits the health data of the user 20 measured by the medical and health equipment 4 (i.e., the health data of the user 20 collected to the collection unit 50 from the medical and health equipment 4) to the health management server 16. The required check item is at least one of the item indicating a worsening health state of the user 20, the item with insufficient measurement data, or the item lacking measurement data.

According to the embodiment, in response to receipt of the request from the health management server 16, the measurement control unit 53 displays the message prompting the user 20 to take a measurement of the required check item. Further, the acquired health data is stored in bulk in the health management server 16, enabling the managerial or related party to check the stored information as necessary.

Thereby, the user 20 is guided to appropriately take a measurement of the health data item relevant to the health maintenance of the body of the user 20. Further, with the acquired health data stored in bulk, the managerial or related party is able to check the stored information as necessary.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA), and conventional circuit components arranged to perform the recited functions. Further, the above-described steps are not limited to the order disclosed herein.

1. A health data management system comprising:
a health data measurement apparatus including
medical and health equipment configured to measure health data related to a body of a user using the medical and health equipment, and
first circuitry configured to collect the health data of the user from the medical and health equipment; and
a health management server communicable with the health data measurement apparatus, the health management server including
a first memory configured to store the health data of the user from the health data measurement apparatus, and
second circuitry configured to
based on the health data of the user stored in the first memory, generate health management data representing a health state of the user,
based on the generated health management data, analyze at least one of an item indicating a worsening health state of the user, an item with insufficient measurement data, or an item lacking measurement data as a required check item,
based on the analysis, determine whether the health management data includes the required check item, and
based on a determination that the health management data includes the required check item, transmit to the health data measurement apparatus a message prompting the user to take a measurement of the required check item, and request the health data measurement apparatus to transmit the health data of the user collected from the medical and health equipment to the health management server.

2. The health data management system of claim 1, wherein the health data measurement apparatus further includes a display, and
wherein in response to receipt of a request from the health management server, the first circuitry controls the display to display the message prompting the user to take a measurement of the required check item, and transmits the health data of the user collected from the medical and health equipment to the health management server.

3. The health data management system of claim 2, wherein the health data measurement apparatus further includes a second memory configured to store an operation manual of the medical and health equipment, and
wherein when the message is displayed on the display, the first circuitry controls the display to display the operation manual.

4. The health data management system of claim 2, wherein the health data measurement apparatus further includes a printer and a second memory configured to store an operation manual of the medical and health equipment, and
wherein when the message is displayed on the display, the first circuitry controls the printer to print the operation manual.

5. The health data management system of claim 1, wherein the second circuitry calculates, for each health management data item, a first value of correlation between the generated health management data and standard health management data, and
wherein when the first value of correlation meets a first particular condition, the second circuitry extracts the required check item.

6. The health data management system of claim 1, further comprising an attendance management server configured to manage working hours of the user,
wherein the second circuitry
acquires working hours data of the user from the attendance management server,
calculates, for each health data item, a second value of correlation between the working hours data and the health data, and
when the second value of correlation meets a second particular condition, extracts the required check item.

7. The health data management system of claim 1, further comprising a first terminal operated by medical personnel.

8. The health data management system of claim 1, wherein the health data measurement apparatus displays a display screen that displays the health data as a two-dimensional code,
wherein the health data measurement apparatus further includes a scanner configured to read the displayed two-dimensional code, and
wherein the first circuitry converts the two-dimensional code read by the scanner into the health data.

9. The health data management system of claim 2, wherein the first circuitry
controls the display to display a message prompting the user to place a palm of a hand of the user on a reading surface of a scanner,
issues a read command to the scanner to scan the palm of the hand of the user,
acquires image data of the scanned palm of the hand of the user from the scanner, and
based on the image data, analyzes components contained in biological tissues of the palm of the hand of the user.

10. The health data management system of claim 4, wherein the printer supplies infrared toner to an electrostatic latent image on a photoconductor to form a non-visualized toner image on a recording medium.

11. The health data management system of claim 2, further comprising a second terminal configured to be operated by a health manager,
wherein when the second circuitry fails to acquire health data of the required check item corresponding to the message prompting the user to take the measurement, the second circuitry transmits a message to the second terminal to notify lack of the health data of the required check item.

12. A health data measurement apparatus connected to a health management server via a network, the health data measurement apparatus comprising:
a display;
medical and health equipment configured to measure health data related to a body of a user; and
circuitry configured to
control measurement of the medical and health equipment,
in response to receipt of a request from the health management server, control the display to display a message prompting the user to take a measurement of a required check item, and
transmit the health data of the user measured by the medical and health equipment to the health management server,
the required check item being at least one of an item indicating a worsening health state of the user, an item with insufficient measurement data, or an item lacking measurement data.

13. A health data management method comprising:
storing health data of a user in a memory from a health data measurement apparatus, the health data of the user being collected to the health data measurement apparatus from medical and health equipment;
based on the health data of the user stored in the memory, generating health management data representing a health state of the user;
based on the generated health management data, analyzing at least one of an item indicating a worsening health state of the user, an item with insufficient measurement data, or an item lacking measurement data as a required check item;
based on the analysis, determining whether the health management data includes the required check item; and
based on a determination that the health management data includes the required check item, transmitting to the health data measurement apparatus a message prompting the user to take a measurement of the required check item, and requesting the health data measurement apparatus to transmit the health data of the user collected from the medical and health equipment to the health management server.
